(19) United States
(12) Patent Application Publication
(10) Pub. No.: US 2018/0263226 A1
(43) Pub. Date: Sep. 20, 2018

(54) COMPOSITIONS AND METHODS FOR CREATING PANCREATIC CANCER ANIMAL MODEL

(71) Applicant: Bruno DOIRON, San Antonio, TX (US)

(72) Inventor: Bruno DOIRON, San Antonio, TX (US)

(73) Assignee: THE BOARD OF REGENTS OF THE UNIVERSITY OF TEXAS SYSTEM, Austin, TX (US)

(21) Appl. No.: 15/542,702

(22) PCT Filed: Jan. 18, 2016
(86) PCT No.: PCT/US16/13807

§ 371 Date: Jul. 11, 2017

Related U.S. Application Data
(60) Provisional application No. 62/104,459, filed on Jan. 16, 2015.

(51) Int. Cl. A01K 67/027; A61K 49/00; C12N 15/113; C07K 14/82

(52) U.S. Cl. CPC A01K 67/0275; A61K 49/0008; C12N 15/1135; C07K 14/82; A01K 2227/105; C12N 2310/14; A01K 2217/058; A01K 2267/0331; C12N 2310/531

(57) ABSTRACT

Certain embodiments are directed kits comprising components for producing a mammalian cancer model. In certain aspects the components are expression vectors. In certain embodiments one or more expression vector is engineered to express a Kras^{G12D} polypeptide, a p53 transcriptional suppressor, SMAD4 transcriptional suppressor, p16/CDKN2A transcriptional suppressor. In certain aspects the transcriptional suppressor is a short hairpin RNA (shRNA) or other nucleic acid used for RNA interference.

This application claims priority to U.S. Provisional Application Ser. No. 62/104,459 filed Jan. 16, 2015. This application is incorporated herein by reference in its entirety.

### BACKGROUND

Pancreatic cancer is one of the most intractable cancers to treat and the least understood of all human cancers. Overweight/obesity, as well as diabetes, are risk factors for pancreatic cancer. The relative risk of pancreatic cancer is increased approximately 1.5-fold for obese subjects and 2 to 3-fold for type 2 diabetic patients (Bosetti et al., 2012). The increased prevalence of overweight/obesity and type 2 diabetes mellitus (T2DM) has reached epidemic proportions during the last decade, and this may, in part, explain why mortality from pancreatic cancer has not declined in the same way as lung cancer, cancer of the upper digestive tract, bladder cancer, and other cancers (Bosetti et al., 2012).

Advances in the treatment of pancreatic cancer have been negligible over the last two decades, and survival has not improved (5-yr relative survival<5%) (Sharma et al., 2011). Type 2 diabetes mellitus is the third most modifiable risk factor for pancreatic cancer, after cigarette smoking and obesity (Li, 2012). The current therapy for pancreatic cancer is gemcitabine and nab-paclitaxel or 5-Flurouacil based combination chemotherapy that only extends life by a matter of couple of months.

Oncologic drug development relies heavily on mouse models bearing transplanted tumors to test the efficacy of agents. However, the ability of such models to predict utility of cancer drugs has been disappointing. Transplanted tumors behave quite differently than tumors in situ, and fail to fully recapitulate the behavior of the original malignant cells. Unfortunately, neither cell-based assays nor xenograft models have been particularly successful in predicting drug responses in humans. Transgenic mice (embryonic overexpressed proteins) and embryonic knockout mice do not mimic the pathogenic mechanisms responsible for the development of cancer that appear postnatal. One of the major risk factors for cancer is aging, not the corpus development stage. There remain significant differences between cancer development in humans and in genetically engineered mouse models (Rangarajan and Weinberg, 2003). Both knockout models and overexpression of mutated proteins in transgenic mice have proven useful for modeling hereditary tumor syndromes that affect embryonic development. However, they are not well suited to the study of spontaneous cancers.

Conditional recombinant transgenic mice with drug-sensitive promoter elements can be used to achieve time-dependent expression of oncogene to induce tumors after development, i.e., as adults. However, models relying on a tissue-specific promoters result in the creation of mutated cells surrounded by other mutant cells. In a spontaneous human tumor, the initiating mutation most likely occurs in a cell that is surrounded by normal cells. Studies with tissue-specific promoters to create a pancreatic cancer model has to be interpreted with caution due to the problems similar to that of the using of the pdx-1 promoter to express Cre and the inherent artifacts produced by the 1oxP-flanked DNA segments by Cre recombinase into the pancreas (Schimidt-Supprian and Rajewsky, 2007).

The utilization of pdx1 promoter to express cre in the pancreas not only targets the pancreatic beta cell, but also expresses cre in the brain during development (Honing et al., 2010). These brain regions where recombination of the Pdx1-cre mouse line occurs are well known to have important functions in glucose homeostasis and other pathways (Habener et al., 2005). Pdx1 is expressed in all pancreatic cells during embryonic development and in endocrine cell in post development (Haberner et al., 2005). In adult pancreas, subpopulations of somatostatin-producing and pancreatic polypeptide-producing cells also express Pdx-1, e.g., insulin producing cells (Habener et al., 2005). Furthermore, Pdx1 is expressed in the intestine (Chen et al., 2009). The problematic tissues specific expression exists also when pdx1 promoter is used to overexpress a protein(s) in the mouse model, as a result, not only the pancreatic tissues will be targeted. Therefore, the temporal timing of the deletion of the protein implicated in two different functions in development and post-development would result in a completely different phenotype.

One most important potential problem in conditional gene-disruption experiments using Cre recombinase is Cre toxicity (Schimidt-Supprian and Rajewsky, 2007). While most cre-transgenic mice lines seem to develop normally, a study demonstrated that Cre can be toxic to cells, by damaging genomic DNA resulting from the recombinase activity. As Schimidt-Supprian and Rajewsky (2007) suggest, many Cre-expressing mouse lines are not completely normal but may largely overcome Cre toxicity through developmental selection and adaptation processes. If the Cre toxicity induces a DNA mutation that leads to induced activation of an oncogene, the result would produce misleading outcomes in the creation of a pancreatic cancer model.

There is a need for additional methods and compositions for creating a cancer model, in particular a pancreatic cancer model.

### SUMMARY

Certain embodiments are directed to kits comprising components for producing a mammalian cancer model. In certain aspects the components are expression vectors. In certain embodiments one or more expression vector is engineered to express a Kras^{G12D} polypeptide, a p53 transcriptional suppressor, a SMAD4 transcriptional suppressor, and/or a p16/CDKN2A transcriptional suppressor. In certain aspects the transcriptional suppressor is a short hairpin RNA (shRNA) or other nucleic acid used for RNA interference.

Certain embodiments are directed to methods for creating a mammalian model of a cancer by contacting a target organ in a mammal with a lentivirus expressing a Kras^{G12D} polypeptide (e.g., SEQ ID NO:2). In certain aspects a transcriptional suppressor of one, two, or three of p53 (e.g., SEQ ID NO:3), p16/CDKN2A (e.g., SEQ ID NO:4, 5, and/or 6), and/or SMAD4 (e.g., SEQ ID NO:7) is administered at about the same time as the Kras^{G12D} expressing nucleic acid. In one embodiment the Kras^{G12D} expressing nucleic acid also comprises an expression cassette for one, two, or three of the transcriptional suppressors. In a further embodiment a single vector encodes the Kras^{G12D} encoding sequence as well a p53, p16/CDKN2A, and/or SMAD4 transcriptional suppressor. The transcriptional suppressor can be a shRNA. In certain aspects the Kras^{G12D} polypeptide is encoded by a first nucleic acid, and one or more transcriptional suppressor is encoded by a second distinct nucleic acid. In one aspect all three transcriptional suppressors are expressed from a single nucleic acid. In one aspect the expression vector is a Lentiviral expression vector.

In certain aspects nucleic acids are delivered to the pancreas by placing a catheter in the body of a subject and locating the tip of the catheter in the pancreas. Once the catheter is in an appropriate location nucleic acid vectors can be introduced by injection through the catheter. In certain aspects one or more nucleic acid vector is delivered to the pancreas via the pancreatic duct. In certain aspects the target organ is the pancreas and the cancer is pancreatic cancer.

In certain aspects the mammal is a rodent (mouse, rat, etc.), a non-human primate (chimp, baboon, monkey, etc.) or an ungulate (horses, cattle, sheep, goats, etc.).

Certain embodiments are directed to assays for or methods of identifying anti-cancer compounds or therapies. The methods for identifying anti-cancer therapies include administering a test or candidate compound to an pancreatic cancer animal model as described herein and determining the therapeutic effect of the test of candidate compound by a monitoring and evaluating the progression, inhibition, or amelioration of pancreatic cancer in the animal model. In certain aspects the effect is relative to a non-therapeutic control and/or in comparison to a known therapeutic. A number of measure of effectiveness can be used and include but are not limited to tumor size, prolonged survival of the animal model, or the reduction in cancer cells or cancer cell mass.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Bioluminescence image of 3 weeks post-injection of a cocktail of Lentivirus shRNA p53 combine with Lentivirus Kras^{G12D} (Image taken 10 min after i.p. administration of luciferin; 60 second acquisition, binning 10). Both lentivirus Kras^{G12D} and shRNA p53 co-express luciferase gene for tumor detection by the Xenogen IVIS system, which allows one to look at tumors with bioluminescence in vivo. Briefly, a 32-gauge catheter (Braintree Scientific) was inserted into the cystic duct through a small opening on the bottom of the gallbladder. The catheter was then advanced into the common bile duct and secured in place with a slipknot of 0/0 suture around the bile duct and catheter to prevent vector reflux into the liver. With a micro clamp placed around the sphincter of Oddi to avoid leakage of the vector into the duodenum, 50 μl of each Lentiviral vector, shRNA p53 and Kras^{G12D}, co-expressing luciferase was slowly injected into the pancreatic duct through the catheter. The bioluminescence image demonstrated that only the pancreas has been specifically targeted with our in vivo method of injection. Male mice CFW (Swiss Webster) eight weeks old on the day of Lentivirus shRNA p53 and Kras^{G12D} injection were used.

FIG. 2. Pancreatic tumor develops spontaneously as pancreatic cancer in adult wild-type mice (Species strain: CFW Swiss Webster) after 28 weeks post-injection of the intraductal cocktail lentivirus-shRNA p53 and Lentivirus-Kras^{G12D}. Both lentivirus Kras^{G12D} and shRNA p53 co-express the luciferase gene for tumor detection by the Xenogen IVIS system, which allows visualization of the pancreatic tumor with bioluminescence in vivo. The in vivo visualization of pancreatic tumor by bioluminescence progressed exponentially for 28 weeks post-injection to 30-week post-injection. FIG. 3 demonstrates using an open incision through the abdomen and the tumor developing in the pancreatic tissues after 30-week post-injection with lentivirus cocktail Kras^{G12D} and shRNA p53. FIG. 4 demonstrates by western blot on pancreatic tissues that the overexpression of specific pancreatic cancer marker Kras, cytokeratin 7/17 and cytokeratin 18 after 30 weeks post-injection of cocktail Kras^{G12D} and shRNA p53. These data validate the composition and method for creating a new pancreatic cancer animal model that is desperately needed to replace the non-predictable mice models that are available today in pancreatic cancer to test medicine and study the disease.

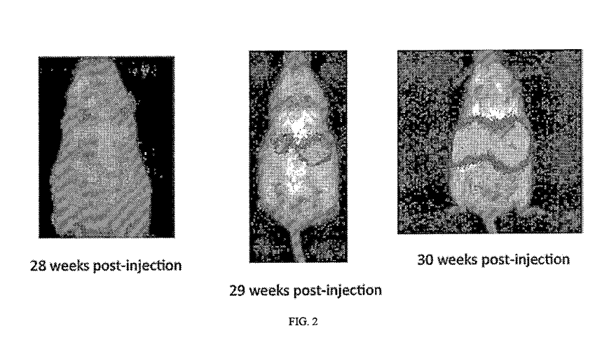

FIG. 3 Spontaneous Pancreatic Cancer in Adult Wild-Type Mice injected directly into the pancreas with lentivirus-shRNA p53 and lentivirus-Kras^{G12D}. Tumor localized specifically to the pancreatic tissues with open abdominal surgery by bioluminescence image.

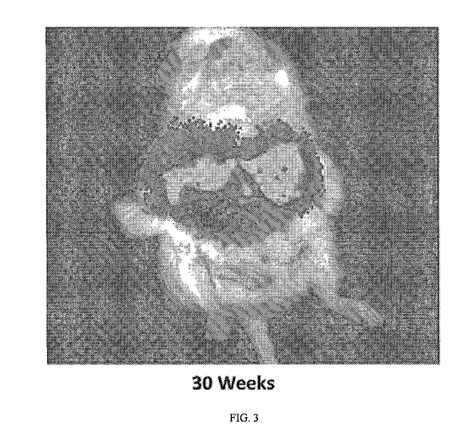

FIG. 4 Spontaneous Pancreatic Cancer in Adults Wild-Type Mice injected directly into the pancreas with lentivirus-shRNA p53 and Lentivirus-Kras^{G12D}. Pancreatic cancer Kras oncogene and cytokeratin 7/17 and 18 Tumor marker Pancreatic Tissues Western Blot.

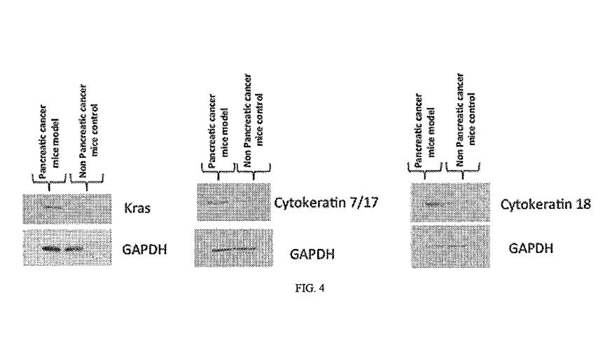

FIG. 5 Spontaneous Pancreatic Cancer in Adults Wild-Type Mice injected directly into the pancreas with lentivirus-shRNA p53 and lentivirus-Kras^{G12D}. Histology of pancreatic tissue demonstrated developing tubular complex and surrounding fibroblasts characteristic of pancreatic cancer. Histology showed the formation of tubular structure with both ductal and acinar differentiation that replaces acinar parenchyma in pancreatic cancer.

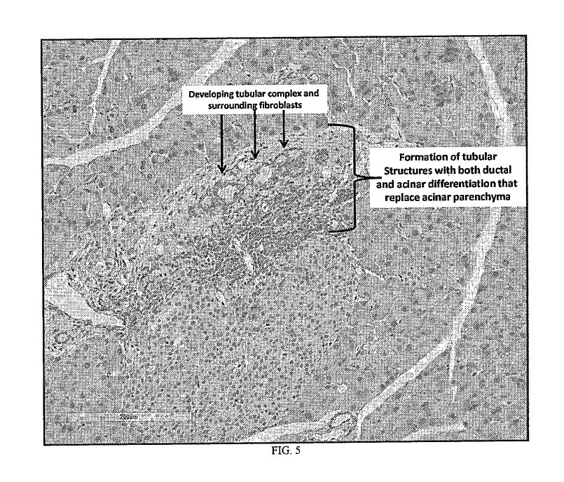

FIG. 6 Spontaneous Pancreatic Cancer in Adults Wild-Type Mice injected directly into the pancreas with lentivirus-shRNA p53 and lentivirus-Kras^{G12D}. Pancreatic tissues showed development of Pancreatic Intraepithelial Neoplasia (PanINs).

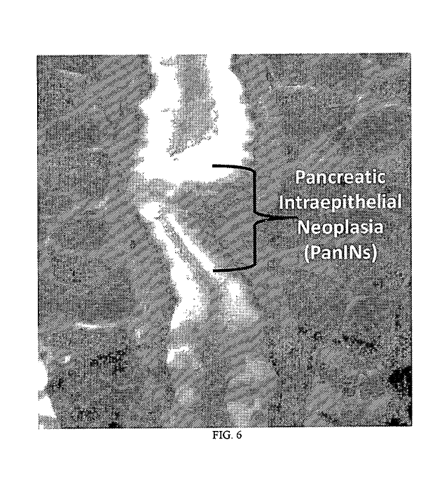

FIG. 7 Spontaneous Pancreatic Cancer in Adults Wild-Type Mice injected directly into the pancreas with lentivirus-shRNA p53 and lentivirus-Kras^{G12D}. Periodic Acid-Shiff (PAS) stains shown acinar cell carcinoma development.

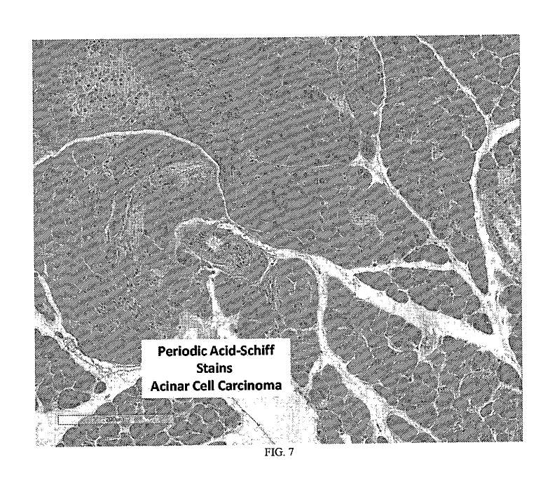

FIG. 8 Spontaneous Pancreatic Cancer in Adults Wild-Type Mice injected directly into the pancreas with lentivirus-shRNA p53 and lentivirus-Kras^{G12D}. Histology showed carcinomas that are fully invasive in pancreatic tissue. The histology showed an acinar cells carcinoma with solid patterns uniform round nuclei.

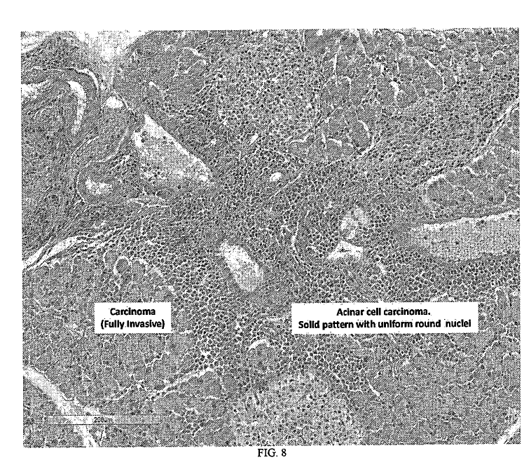

FIG. 9 Spontaneous Pancreatic Cancer in Adults Wild-Type Mice injected directly into the pancreas with lentivirus-shRNA p53 and lentivirus-Kras^{G12D}. Histology is showing p-ERK activities in pancreatic ductal cells. ERK signaling pathway plays an important role in pancreatic cancer development and progression by contributing to cell cycle regulation, differentiation, proliferation, survival and migration.

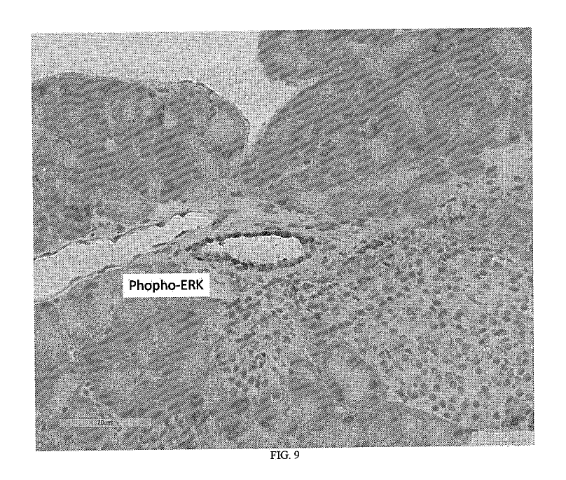

### DESCRIPTION

Most (˜95%) pancreatic cancers arise in the exocrine pancreas (Anderson et al., 2006). The endocrine tissues account for only about 2% of the volume of the pancreas. Oncogenes can be activated through a variety of mechanisms, including point mutations within the gene and amplification of the gene itself. A growing number of oncogenes have been identified in pancreatic cancer. The most common activating point mutation involves the Kras oncogene (present in over 90% of human pancreatic cancers) (Almoguera et al., 1988). Oncogenic mutation of Ras remains constitutively active in the GTP-bound form with impaired GTPase activity. Activation of Ras induces cell cycle progression. In addition to activation of Kras, the tumor suppressor p53 is frequently inactivated in human pancreatic cancers (Barton et al., 1991).

The TP53 tumor-suppressor gene on chromosome 17p encodes for the p53 protein (Redston et al., 1994). The TP53 gene is inactivated in 55-75% of pancreatic cancers (Redston et al., 1994). Because of the loss of TP53, the mechanisms involved in the regulation of cell division and cell apoptosis are deregulated in the majority of pancreatic cancers. The TP53 gene mutation is a late genetic event in pancreatic cancer progression (Moore et al., 2001). In contrast, the Kras mutation is an early event in pancreatic cancer progression (Almoguera et al., 2006). However, the Kras mutation itself is not sufficient to develop invasive pancreatic cancer. In the knockout mouse model, TP53 signaling, in the absence of oncogenic Kras, does not lead to the development of pancreatic carcinoma (Koorstra et al., 2008). However, in an embryonic genetically engineered mouse model, when mutations for both Kras and TP53 are present, pancreatic cancer develops (Koorstra et al., 2008). Therefore, shRNA targeting p53 protein and overexpression of Kras mutation of the constitutively active form is used to create a post-embryonic/post-developmental pancreatic cancer model in mice that recapitulates many aspects of the human disease. This allows the testing of the therapeutic efficacy of drug(s) in a mouse model that reflects the genetic and post-embryonic development of human pancreatic cancer.

A mouse model of pancreatic cancer should reflect the human disease and include both the genetics of the targeted cells, as well as surrounding non-targeted cells. The ideal model should produce subtle, controlled mutations in relevant endogenous genes in targeted cells, while leaving an effectively wild-type genotype in non-targeted cells. Injection of the lentiviral vector in vivo creates the perfect model for pancreatic cancer by transferring genetic material randomly in the tissues and creating mutant cells surrounded by non-mutant cells. An advantage of lentivirus vectors is that they do not activate dendritic cells, which are activated by the adenovirus vector. Furthermore, lentivirus vectors can infect and integrate into both dividing and non-dividing cells, they provide high transduction efficiency and sustained gene expression in vivo, they do not induce a significant host immune response and, in contrast to adenovirus vectors, lentivirus vectors can be successfully re-administered (Doiron et al., 2012; Kafri et al., 1997). Consistent with this, lentivirus does not induce inflammation (the hallmark of an immune response) at the site of injection (Kafri et al., 1997). A very important characteristic of the lentivirus is that it does not induce any inflammation at the site of the injection, therefore not interfering in the tissues histological analysis. The method of viral vector injection in vivo into an adult murine pancreas (Doiron et al., 2012) permits one to recreate the spontaneous human pancreatic cancer that appears in post-developmental stage. Using this technique, which allows delivery of the lentiviral vector specifically into the mouse pancreas in vivo (Doiron et al., 2012), a new model of pancreatic cancer is developed.

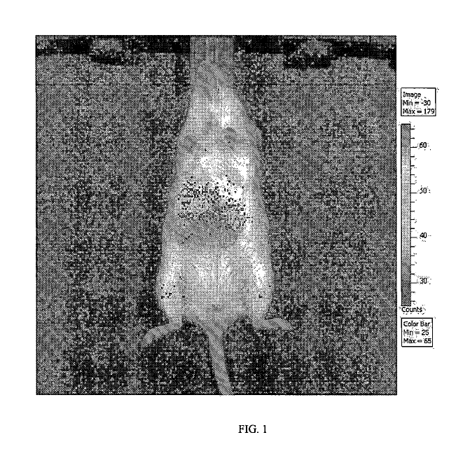

Sixty-eight of 82 (83%) human pancreatic carcinomas have been shown to harbor an activating point mutation in codon 12 of the Kras oncogene (Hruban et al., 1993). The most frequent mutation found in Kras is a guanine to adenine transition (GGT→GAT); this mutation is incorporated into cDNA Kras plasmid construct used. The QuickChange™ Site-Directed Mutagenesis kit (Stratagene Cloning Systems, La Jolla, Calif.) was used to incorporate the Kras mutation (GGT→GAT) into a lentiviral vector under control of the promoter cytomegalovirus (CMV) (Doiron et al., 2012). Lentiviral vector shRNA targeting the p53 protein under the control of promoter U6 was made, as previously described (Doiron et al., 2012). The polymerase III promoter U6 is active ubiquitously in all cells, because of the housekeeping function of polymerase III. Both lentivirus Kras^{G12D} and shRNA p53 co-express the luciferase gene for tumor detection by the Xenogen IVIS system, which allows looking at tumors with bioluminescence in vivo (FIG. 1). The two lentivirus vector oncogene(s) (50 μl each at 1×10⁸ TU/ml) have been injected together in 8-week old adult mice to directly target the pancreas, as previously described (Doiron et al., 2012). A control group injected with lentivirus shRNA scramble and lentivirus GFP co-expressing luciferase protein is compared with the experimental group.

Tumor growth was followed with the Xenogen IVIS imaging system (Xenogen Corp. Alameda Calif.). With spontaneous cancer mouse models, it often is unclear which animals have developed overt tumors. Simply choosing a time point at which to enroll the animals would result in treating numerous mice that do not harbor tumors and necessitate the use of a much larger animal cohort. This study is based on a time point at which the presence of tumors has been detected with bioluminescent, using the luciferase reporter system in combination with Xenogen IVIS imaging (Xenogen Corp, Alameda, Calif.). Imaging modalities allow animals to be studied with tumors of a particular size and location, and this reduces the variability between animals. Using bioluminescent, whether the pancreatic cancer has spread to distant organs is evaluated.

Pancreatic tumor progression has been evaluated in vivo and at post-mortem with RT-PCR markers and histological analysis of the pancreatic cancer. The tumor progression in vivo is evaluated with bioluminescence using the Xenogen IVIS imaging system. Images can be taken 10 minutes or longer after i.p. injection of luciferin (225 mg/kg; Xenogen Corp.) using a 60-second acquisition period. During image acquisition, mice are sedated continuously via inhalation of ˜3% isoflurane (Abbott Laboratories Ltd, Kent, UK). Ex vivo bioluminescence imaging of the isolated pancreas are performed immediately after euthanasia of the animal with CO₂, 10 min after i.p. injection of luciferin, as described above. Image analysis and bioluminescent quantification has been performed using Living Image Software (Xenogen Corp.). Tumor growth from mice injected (n=10) with Lenti-shRNA p53 and Lenti-Kras^{G12D} has been evaluated for more than one year. Kras mutations induce pancreatic intraepithelial neoplastic (PanIN) lesions similar to those observed in humans and these neoplastic lesions express the duct marker cytokeratin-19 (CK19) and produce periodic acid-Schiff (PAS)-reactive mucins. The PanIN scheme for classifying these lesions represents the gold standard at academic centers worldwide (Hruban et al., 2001). PanINs are microscopic lesions in the smaller (less than 5 mm) pancreatic ducts and are classified into a four tiers: PanIN-1A, PanIN-1B (low-grade PanINs), PanIN-2 (intermediate grade PanINs) and PanIN-3 (high-grade PanIN), reflecting a progressive increase in histologic grade culminating in invasive neoplasia.

Gross evaluation of the pancreas includes careful documentation of the size, shape, consistency, and color of the gland, as well as the localization, size, and gross appearances of any lesions. Other organs, especially liver and intestine, are inspected for evidence of metastases or other pathology. The microscopic evaluation of the pancreas determines which cellular compartments (acinar, ductal, endocrine, and interstitial compartments) are affected. Architectural changes alter the relationships among cells or the organization of a compartment. Architectural changes include (a) formation of new abnormal elements including mass (solid masses, papillae, cysts, etc.), (b) an aberrantly located compartment within the pancreas (e.g., ducts within an islet of Langerhans), (c) transformation within preexisting units (cystic change within ducts, etc.), and (d) infiltration by cells not normally found in the gland (inflammatory infiltrates, secondary tumors, etc.). The description of cytological changes includes (a) hypertrophy/atrophy, (b) hyperplasia, (c) metaplasia, (d) proliferation-increase mitoses, (e) atypia, and (f) cell death (apoptosis or necrosis). Specific interstitial changes would include (a) desmoplasia (b) active (cellular) fibrosis, (c) inactive (hypocellular) fibrosis, (d) inflammation, and (e) vascular alterations.

Blood analyses: Following an overnight fast, tail vein blood sample are taken for insulin and glucagon measurement (Doiron et al., 2012). Fasting plasma insulin is measured by using 5 μl (EDTA) samples from the tail vein using the mouse insulin Ultrasensitive ELISA (Alpco Diagnostics, Salem, N.H.). Fasting plasma glucagon concentration is determined from 50 μl plasma samples (EDTA, aprotinin 500 kIU [Trasylol; Bayer HealthCare, Pittsburgh, Pa.] for every 1 ml of blood) using a mouse glucagon ELISA assay (Alpco Diagnostics). The insulin and glucagon level is measured for indication of endocrine tumor's formation. Pancreatic lipase and amylase level measurement use the lipase detection kit (Colorimetric assay, ab102524) and amylase assay kit (ab102523) form Abcam, Cambridge, Mass. Aspartate Aminotransferase (AST) kit MAK055 and Alanine Aminotransferase (ALT) kit MAK052 colorimetric assay form Sigma-Aldrich is used.

Western blot: Equal amounts of total protein is separated on a 10% and 15% SDS/PAGE and transferred onto nitrocellulose membranes. Membranes are blocked with 5% non-fat milk in 0.1% Tris buffered saline (TBS) Tween-20 and are probed with specific antibodies against tumors markers listed in the next immunohistochemical analysis section. Membranes are then incubated with horseradish peroxidase-conjugated secondary antibody (NA934) and are developed with a chemiluminescent reagent (Amersham Bioscience, GE Healthcare, Pittsburgh, Pa.) (Doiron et al., 2012).

Immunohistochimical analysis: The mouse pancreatic tissue is fixed by immersion in phosphate buffer, 4% paraformaldehyde, 1% glutaraldehyde overnight at 4° C. and subsequently embedded with tissues—Tek OCT compound for cryostat sectioning (Doiron et al., 2012). Caspase-3 cleavage in the cancer tissues and apoptosis is measured using the TUNEL assay to evaluate tumor progression (Doiron et al., 20012). The activity of caspase-3 is measured with an antibody (Cell Signaling Technology, Danvers, Mass.) that detects endogenous levels of the large fragment (17/19 KDa) of activated caspase-3 resulting from cleavage nest to Asp175. TUNEL assay is measured with the in situ Cell Death Detection Kit, TMR Red (Roche Diagnostics, Indianapolis, Ind.) PanIN lesions stain strongly with PAS, whereas normal ducts remain unstained. Ex vivo imaging and/or histology is used to confirm and localize metastatic lesions in the pancreas that initially were detected by in vivo imaging. After euthanasia, the pancreas is dissected, a segment is frozen for subsequent analyses, and a segment is prepared for paraffin sections (Doiron et al., 2012). PAS staining is preformed according to the manufacturer's instructions (Sigma-Aldrich). For in vivo BrdU labeling, animals are injected with BrdU (100 mg/kg body weight) intraperitoneally 3 h prior to sacrifice. Immunostaining for BrdU is performed using a monoclonal anti-BrdU antibody (Abcam, Cambridge, Mass.). Labeling indices are calculated as fraction of BrdU-positive cell in relation to the total cell number and are expressed as percentage±SEM. Seven randomly selected, non-overlapping confocal microscope images are taken from Ki67-stained slide from 5 independent animals for each group. Nuclei positive for Ki67 are counted as actively proliferating cells. Primary antibodies (including anti-cytokeratin 19, anti-amylase, anti-GFP, anti-TGF-β, anti-MUC1, anti-MUC2, anti-MUC5, anti-synaptophysin, anti-chymotrypsin, anti-somatostatin (G-10), anti-Ki67 (M-19), anti-glucagon (K79bB10), anti-insulin A (C-12), anti-BCL2-interacting mediator of cell death (BIM) (M-20), anti-amylase [Santa Cruz Biotechnology, Santa Cruz, Calif.]) and secondary antibodies (fluorescent secondary antibodies including donkey anti-goat-fluorescein, goat anti-mouse-flurorescein, goat anti-rabbit Texas Red and donkey anti-goat Texas Red [Santa Cruz]) are used (Doiron et al., 2012). The Olympus FV-1000 laser scanning confocal microscope is used for the histological analysis (Doiron et al., 2012). The quantified of cell proliferation, TUNEL assay, tumor size uses Image J (National Institutes of Health, Bethesda, Md.), (Doiron et al., 2012).

RNA extraction and real-time PCR: Total RNA is extracted from frozen pancreatic tissue using TRIzol reagent (Invitrogen, Carlsbad, Calif.) with Qiagen RNeasy (Qiagen, Valencia, Calif.). The integrity of each RNA sample is confirmed post-extraction using denaturing (glyoxal) agarose gel electrophoresis. Reverse transcription is carried out with 0.5 μg total RNA using the ImProm II reverse transcription system (Promega, Madison, Wis.). Real-time quantitative PCR is performed using 2 μg cDNA with a primer and 5′-terminal 6-carboxyfluorescein (FAM)-labelled TaqMan probe mix from Applied Biosystems (assay ID Hs00193409; Foster City, Calif.) (Doiron et al., 2012). Relative expression values are calculated from a standard curve, sample of each cDNA, and they are normalized to actin RNA. RT-PCR for pancreatic cancer markers include: three tumor-suppressor genes. (p16INK4A/CDKN2A, TP53, and DPC4/SMAD4/MADHA) and the apomucins (MUC1, MUC2, MUC5). RT-PCR is also performed for the protein cyclin D1, Notch pathway receptor (Notch 1-4), and transcriptional targets (Hes 1) that are up-regulated in pancreatic cancer. In vivo tumor bioluminescence is quantified in all animals weekly and the mean photons/second is calculated for the control mice and experimental groups. Endpoint criteria following of the mouse pancreatic cancer model includes the development of abdominal ascites, severe cachexia, significant weight loss exceeding 20% of initial weight, or extreme weakness or inactivity.

The validity of the mouse model is tested by treating the mice with gemcitabine based chemotherapeutic regimen, to examine whether pancreatic cancer model described above, responds similarly to pancreatic cancer in humans. Gemcitabine is a fluorine-substituted dexoycytidine analog that enters into the cell mainly via human equilibrative nucleoside transporter-1. Inside the cell, gemcitabine is activated by the deoxycitidine kinase enzyme and inactivated by cytidine deaminase. The active form of gemcitabine inhibits the enzyme ribonucleotide reductase leading to a decreased level of deoxyribonucleotides essential for DNA synthesis (Strimpakos et al., 2008). Despites gemcitabine's only modest single agent clinical efficacy, novel therapeutic agent(s) in development have been combined with the gemcitabine backbone over the last decade in attempt to further improve efficacy, most if not all have been negative studies (Mahalingam and Giles 2008; Mahalingam et al., 2009; PMID:18851695). These disappointing trials are also and indirect consequence of the lack of convenient mouse models that mimic human cancer well enough to allow the identification on new targets and the testing of new compound against pancreatic cancer.

Two combination options exist as front line therapy for patients with advance pancreatic cancer. Gemcitabine in combination with nab-paclitaxel and 5-Flurouracil regimen in combination with oxaliplatin and/or irinotecan. Nab-paclitaxel is an albumin-bound paclitaxel. Paclitaxel was found to inhibit the depolymerization of microtubules, a process necessary for normal cell division. Specifically, microtubule stabilization blocks cells in G2 and M phase of the cell cycle resulting in cell death (Long and Fairchild, 1994). Nab-paclitaxed is a novel formulation of paclitaxel that binds to SPARC in the tumor microenvironment in pancreatic cancer (Damascelli et al., 2001). 5-Flurouracil widely used in the treatment of cancer. The mechanism of cytotoxicity of 5-Flurouracil has been ascribed to the misincorporation of fluoronucleotides into RNA and DNA and to the inhibition of the nucleotide synthetic enzyme thymidylate synthase (Longley et al., 2003). Oxaliplatin reach with DNA, forming mainly platinated intrastrand crosslinks with 2 adjacent guanines or adjacent guanine/adenine residues and inhibits DNA synthesis (Saris et al., 1996). Irinotecan inhibits topoisomerase I, thus impeding DNA uncoiling leading to double-stranded DNA breaks (Hsiang et al., 1985).

The impact of the treating agent(s) on the tumor and tumor microenvironment is studied as described above by analyzing gross evaluation of the pancreas; architectural changes and specific interstitial changes. These treatment agents like nab-paclitaxel have been suggested depleting the stromal barrier in the tumor microenvironment and thus augmented circulation through the tumor, thereby allowing gemcitabine greater access to tumors.

Mice were compared with a pancreatic cancer group not receiving treatment agent(s). Mice groups based on treatment agent received, (a) Gemcitabine; (b) Gemcitabine and nab-paclitaxel; (c) 5-Flurouracil; (d) 5-Flurouracil and oxaliplatin; (e) 5-Flurouracil and irinotecan

The time for preclinical trial enrollment is determined by the bioluminescence image that possesses tumors that are the same size and located within the pancreas without metastases. This approach allows the growth of the tumor to be tracked before and during treatment. The mice enrollment is at the same age to define the survival curve. The bioluminescence image is used to track tumor progression, stabilization, or remission. This approach is similar to that used in clinical oncology where each mouse acts as an individual cancer patient and yields all the same information to the oncologist. nab-paclitaxel for injectable suspension is obtained from Albraxis BioScience, Inc (Los Angeles, Calif.). The drugs are reconstituted in saline, prepared fresh daily, and given within 1 hour of preparation. nab-paclitaxel alone (10 or 30 mg/kg, intravenously [i.v.] by the tail vein in mice), daily for five consecutive days. Gemcitabine is purchased by Eli Lilly and will be dissolved in buffered saline before administration. Gemcitabine is administered i.p. in a daily schedule for a month. The Gemcitabine treatment begins at the day of mice enrollment using gemcitabine 100 mg/kg on days 0, 3, 6, and 9. Control group receives sham injection with vehicle alone (saline serum). Mice are injected with a single i.p. dose of 150 mg/kg 5-Fluroracil (Roche Laboratories, Nutley, N.J.) on day one of enrollment. Oxaliplatin (lyophilized powder) by Sanofi-Synthelabo is dilute in glucose solution 5% and delivery in a volume of 32 ml/kg by i.p. in mice. Oxaliplatin consists of two administrations every 4 days. Irinotecan (CPT-11; Camptosar, Upjohn) is administrated by i.p. (50 mg/kg) to mice three times at four-day intervals.

The p16-Ink4A locus is encoded by the CDKN2A tumor suppressor gene. The p16 tumor suppressor protein acts to inhibit the binding of the D-family cyclins to their respective cylin-dependent kinase (CDK) partners. Loss of functional p16 protein can result in increased phosphorylation of the Retinoblastoma protein, and therefore lead to increased cell cycle progression through G1 phase into S phase (Schutte et al., 1997). The mutations of p16/CDKN2A gene associated with familial melanoma have been shown to increase risk of pancreatic cancer. Individual from familial melanoma kindreds have a 13- to 22-fold increased risk of developing pancreatic cancer (Lynch et al., 2008) and individuals who carry p16/CDKN2A mutation have a 38-fold increased risk of developing pancreatic cancer compared to the general population (Rutter et al., 2004). A shRNA targeting p16/CDKN2A is incorporated into the lentiviral construct to be injected into the pancreas in combination with the two others lentiviral vector Kras^{G12D} and shRNA p53 to create a synergistic effect of multiple's oncogene(s) that mimic human polygenetic mutation signature of pancreatic cancer.

The transforming growth factor-β (TGF-β) signaling pathway is another important and commonly deregulated signaling pathway in pancreatic carcinomas. Although multiple gene mutations in the TGF-β pathway have been identified, the most common mutation is that of SMAD4 (Jones et al., 2008). The SMAD4 gene, also known as DPC4, is located on chromosome 18q21. SMAD4 inactivation is present in more than half of all pancreatic tumors (Hahn et al., 1996). A shRNA targeting SMAD4 is incorporated into the lentiviral construct to be injected into the pancreas in combination with the two others lentiviral vector Kras^{G12D} and shRNA p53 to create a synergistic effect of multiple's oncogene(s) that mimics human polygenetic mutation signature of pancreatic cancer.

In certain embodiments expression of oncogenic agents are used to create mouse models of pancreatic cancer. In certain aspects the oncogenic agents are encoded in and expressed by nucleic acid vectors. In a further aspect the nucleic acid vectors are viral vectors. In still a further aspect the viral vector is a Lentiviral vector. In certain aspects the oncogenic agent is a Kras^{G12D} protein and a p53 transcriptional or translational suppressor, for example an shRNA p53. In other aspects the oncogenic agents are Kras^{G12D} polypeptide and a p16/CDKN2A transcriptional or translational suppressor, for example an shRNA p16/CDKN2A. In a further aspect the oncogenic agents are Kras^{G12D} polypeptide and a SMAD4 transcriptional or translational suppressor, for example an shRNA SMAD4.

In certain aspects 1, 2, 3, or 4 nucleic acid vectors are configured to express one or more oncogenic agents including (i) Kras^{G12D}, shRNA p53 and shRNA p16/CDKN2A; (ii) Kras^{G12D} shRNA p53 and shRNA SMAD4; or (iii) Kras^{G12D}, shRNA p53, shRNA SMAD4 and shRNA p16/CDKN2A.

Control group mice are injected with the same concentration and volume of the various combinations of lentivirus oncogene(s) of Lentivirus expressing green fluorescence protein and shRNA scramble into the pancreas.

CMV and U6 promoters were used to express different oncogene(s) that target all types of pancreatic cells (endocrine cell, acinar cell and ductal cell). Alternatively, specific pancreatic cell type promoter is inserted into our lentiviral vector(s) to express different oncogene(s) to target only the ductal or acinar cells. The expression of the oncogene(s) under the control of the CK19 ductal promoter is used to specifically target the ductal cell. The acinar-specific promoter (elastase) is used to specifically target the acinar-cells

1. A kit for producing a mammalian cancer model comprising one or more nucleic acid vectors configured to express, individually or in combination one or more of (i) a Kras^{G12D} polypeptide, (ii) a p53 transcriptional suppressor, (iii) a SMAD4 transcriptional suppressor, and/or (iv) a p16/CDKN2A transcriptional suppressor.

2. The kit of claim 1, wherein the transcriptional suppressor is a short hairpin RNA (shRNA).

3. The kit of claim 1, wherein the nucleic acid vectors are Lentiviral vectors.

4. A method for producing a mammalian model of a cancer comprising contacting a target organ in a mammal with a lentivirus expressing a Kras^{G12D} polypeptide and at least one Lentiviral vector expressing a transcriptional suppressor of one, two, or three or p53, p16/CDKN2A, and/or SMAD4.

5. The method of claim 4, wherein the transcriptional suppressor is a shRNA.

6. The method of claim 4 wherein a single Lentiviral vector expresses a Kras^{G12D} polypeptide, a transcriptional suppressor p53, a transcriptional suppressor of p16/CDKN2A, and a transcriptional suppressor of SMAD4.

7. The method of claim 4, wherein the contacting of the organ with the nucleic acid vectors is via a catheter positioned within the organ.

8. The method of claim 7, wherein the nucleic acid vectors are delivered via the pancreatic duct.

9. A method of identifying an anti-cancer therapy comprising administering a candidate compound or therapeutic regime to an animal model produced by the method of claim 4 and assessing the effectiveness of the compound or regime in treating pancreatic cancer in the animal model.


SEQUENCE LISTING (7 sequences)

SEQ ID NO: 1 - DNA, 567 bp, Mus musculus
  CDS (1)..(567)
atgactgagtataagcttgtggtggttggagctgatggcgtaggcaagagcgccttgacgatacagctaattcagaatcactttgtggatgagtacgaccctacgatagaagactcctacaggaaacaagtagtaattgatggagaaacctgtctcttggatattctcgacacagcaggtcaagaggagtacagtgcaatgagggaccagtacatgagaactggggagggctttctttgtgtatttgccataaataatactaaatcatttgaagatattcaccattatagagaacaaattaaaagagtaaaggactctgaagatgtgcctatggtcctggtagggaataagtgtgatttgccttctagaacagtagacacgaaacaggctcaggagttagcaaggagttacgggattccgttcattcagacctcagcaaagacaagacagggtgttgacgatgccttctatacattagtccgagaaattcgaaaacataaagaaaagatgagcaaagatgggaagaagaagaagaagaagtcaaggacaaggtgtacagttatgtga

SEQ ID NO: 2 - protein, 188 aa, Mus musculus
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQELARSYGIPFIQTSAKTRQGVDDAFYTLVREIRKHKEKMSKDGKKKKKKSRTRCTVM

SEQ ID NO: 3 - DNA, 19 bp, Artificial Sequence
  synthetic oligonucleotide
gtactctcctcccctcaat

SEQ ID NO: 4 - DNA, 19 bp, Artificial Sequence
  synthetic oligonucleotide
gaacatggtgcgcaggttc

SEQ ID NO: 5 - DNA, 19 bp, Artificial Sequence
  synthetic oligonucleotide
gaggaggtgcgggcgctgc

SEQ ID NO: 6 - DNA, 19 bp, Artificial Sequence
  synthetic oligonucleotide
ggcagtaaccatgcccgca

SEQ ID NO: 7 - DNA, 21 bp, Artificial Sequence
  synthetic oligonucleotide
gcatagtttgatgtgccatag